(19) United States
(12) Patent Application Publication
(10) Pub. No.: US 2015/0142369 A1
(43) Pub. Date: May 21, 2015

(54) PREDICTION OF CALIFORNIA BEARING RATIO OF SUBBASE LAYER USING MULTIPLE LINEAR REGRESSION MODEL

(71) Applicants: Mohammad Hasan Alawi, Makkah (SA); Maher Ibrahim A. Rajab, Makkah (SA)

(72) Inventors: Mohammad Hasan Alawi, Makkah (SA); Maher Ibrahim A. Rajab, Makkah (SA)

(73) Assignee: UMM AL-QURA UNIVERSITY, Makkah (SA)

(21) Appl. No.: 14/081,137

(22) Filed: Nov. 15, 2013

(51) Int. Cl. G06F 17/18; G01N 33/24; G01N 9/36

(52) U.S. Cl. CPC G06F 17/18; G01N 9/36; G01N 33/24
USPC 702/137

(57) ABSTRACT

A method for predicting the California Bearing Ratio of a pavement subbase layer, wherein samples are collected from different regions of the subbase layer, the samples are tested to determine at least moisture content and density. Each sample is prepared at optimum moisture content and at different densities and tested to determine the California Bearing Ratio for each density and to obtain a dataset of variables. A multiple linear regression model is applied to selected variables from the dataset to relate the determined California Bearing Ratio value to the selected variables from the dataset to obtain a predicted value of the California Bearing Ratio of a subbase having comparable variables.

### FIELD OF THE INVENTION

This invention relates generally to a method for predicting or estimating the California Bearing Ratio of the subbase layer in pavement. More particularly, the invention relates to a method for predicting the California Bearing Ratio of a pavement subbase layer by using multiple linear regression models.

### BACKGROUND OF THE INVENTION

Flexible pavements have been the predominant type of roads used in Saudi Arabia and other parts of the world, where the majority of paved surfaces fall under the overall category of flexible pavements. Flexible pavements may be classified as a conventional or a full depth pavement. Conventional flexible pavements are layered systems that consist of an asphalt mixture (wearing course) over one or more granular layers (base and subbase) which together are constructed over the sub-grade soil. Granular base and subbase layers are essential components of a flexible pavement system where their function is to reduce traffic induced stresses in the pavement structure and to minimize rutting in the base, subbase and subgrades.

All pavement systems are constructed on earth and practically all components are constructed with earth materials. Flexible pavement typically consists of a wearing course bituminous composite built over a base course and subbase resting on a compacted subgrade. The base may be stabilized with either asphalt, cement, lime, or other stabilizers; or untreated using granular material having specific physical properties.

Surface courses usually consist of asphalt or Portland cement concrete. In general, concrete refers to any material consisting of a mixture of aggregates such as sand, gravel, or crushed stone fastened together by cement. Asphalt concrete consists of asphalt cement and aggregate. Base courses normally consist of aggregates such as gravel and crushed rock. These may be compacted or stabilized by lime, Portland cement or asphalt. Subbases are usually local aggregate materials. They may consist of either unstabilized compacted aggregate or stabilized materials.

The subgrade is the top surface of a roadbed upon which the pavement structure and shoulders are constructed. The purpose of the sub grade is to provide a platform for construction of the pavement and to support the pavement without undue deflection that would impact the pavement's performance. The upper layer of this natural soil may be compacted or stabilized to increase its strength.

The California Bearing Ratio (CBR) test is a simple strength test that compares the bearing capacity of a material with that of a well-graded crushed stone. Thus, a high quality crushed stone material should have a CBR of 100. It is primarily intended for, but not limited to, evaluating the strength of cohesive materials having maximum particle sizes less than 19 mm (0.75 in.) (AASHTO, 2000). It was developed by the California Division of Highways around 1930 and was subsequently adopted by numerous states, counties, U.S. federal agencies and internationally. As a result, most agency and commercial geotechnical laboratories in the U.S. are equipped to perform CBR tests.

The basic CBR test involves applying load to a small penetration piston at a rate of 1.3 mm per minute and recording the total load at penetrations ranging from 0.64 mm up to 7.62 mm. The California Bearing Ratio or CBR test is an indirect measure of soil strength based on resistance to penetration by a standardized piston moving at a standardized rate for a prescribed penetration distance. CBR values are commonly used for highway, airport, parking lot, and other pavement designs based on empirical local or agency specific methods (i.e., FHWA, FAA, and AASHTO). CBR has also been correlated empirically with resilient modulus and a variety of other engineering soil properties.

CBR is not a fundamental material property and thus is unsuitable for direct use in mechanistic and mechanistic-empirical design procedures. However, it is a relatively easy and inexpensive test to perform, it has a long history in pavement design, and it is reasonably well correlated with more fundamental properties like resilient modulus. Consequently, it continues to be used in practice.

The Los Angeles (L.A.) abrasion test is a famous test used to indicate aggregate toughness and abrasion characteristics. Aggregate abrasion characteristics are important because the constituent aggregate in hot mixed asphalt (HMA) must resist crushing, degradation and disintegration to produce a high quality mix. This test was conducted according to AASHTO T 96 or ASTM C 131.

Experimental testing of sub-base and sub-grade soils is costly and complex. Other investigators have used artificial neural networks (ANN) for estimating the resilient modulus of sub-base and sub-grade soils from basic material properties and in-situ conditions. See H. I. Park, G. C. Kweon and R. S. Lee, “Prediction of Resilient Modulus of Granular Subgrade Soils and Subbase Materials using Artificial Neural Network”, Road Materials and Pavement Design, Volume 10, Issue 3, January 2009, pages 647-665. This method (ANN) is a reliable and simple predictive tool for estimating the resilient modulus of sub-base and sub-grade materials.

Artificial intelligence (AI) methods have been proposed for the estimation of California bearing ratio (CBR) values in geotechnical engineering. Among the new researches artificial neural network (ANN) and gene expression programming (GEP) were applied for the prediction of CBR of fine grained soils from Southeast Anatolia Region of Turkey. It was found that maximum dry unit weight (yd) is the most effective parameter on CBR among others such as plasticity index (PI), optimum moisture content (wopt), sand content (S), clay+silt content (C+S), liquid limit (1 L) and gravel content (G). See Taskiran, T. (2010), Prediction of California bearing ratio (CBR) of fine grained soils by AI methods, Advances in Engineering Software, 41(6), 886-892

Yildirim et al., 2011, estimated the California Bearing Ratio (CBR) using soft computation (regression analysis and artificial neural network) from sieve analysis, atterberg limits, maximum dry density and optimum moisture content of subgrade soil in Turkey's regions. They found strong correlations (R²=0.80-0.95) between sieve analysis, atterberg limits, maximum dry density and optimum moisture content. They recommended that the proposed correlations will be useful for a preliminary design of a project where there is a financial limitation and limited time. See Yildirim B. and Gunaydin O. (2011), “Estimation of California Bearing Ratio by Using Soft Computing Systems”, Expert Systems with Applications, 38(5), 6381-6391.

Al-Refeal et al, 1997, predicted a CBR value from a dynamic cone penetrometer test of different types of soil ranging from clay to gravely sand. Unique models were found for each type of soil with good coefficient of determination and low standard error of estimate. The combined data also gave a correlation between CBR and penetration depth CD), which compare very well with those obtained from other studies. See Al-Refeal T. and Al-Suhaibani A. (1997), “Prediction of CBR Using Dynamic Cone Penetrometer”, King Saud University Journal, Vol. 9, Eng. Sci. (2), pp 191-204.

U.S. Pat. Nos. 4,106,296, 4,107,112, 5,352,062, 7,455,476, 8,206,059, 8,297,874 and 8,337,117 are exemplary of various soil stabilization and paving methods and systems involving to a greater or lesser extent the California Bearing Ratio, Los Angeles Abrasion test, and/or resilient modulus.

U.S. Pat. No. 4,106,296 to Leonard Jr. et al. discloses a method of soil stabilization for sub-bases with data including sieve analysis, optimum moisture content and California Bearing Ratio percent.

U.S. Pat. No. 4,107,112 to Latta Jr. et al. discloses an epoxy resin soil stabilizing composition and data including sieve analysis, optimum moisture content, maximum dry density and California Bearing Ratio percent.

U.S. Pat. No. 5,352,062 to Yoshida et al. discloses a skid road surface and method of construction with Los Angeles abrasion test for hardness of crushed stone.

U.S. Pat. No. 7,455,476 to Grubba et al discloses a method of reconstructing a bituminous-surfaced pavement with a determined moisture content, cohesion, and modulus test, with percent of aggregate passing through a selected sieve size, and California Bearing Ratio determination.

U.S. Pat. No. 8,206,059 to Southgate et al. discloses a load transfer assembly between concrete slabs, with estimate of subgrade modulus from the soil California Bearing Ratio by formula.

U.S. Pat. No. 8,297,874 to Krzyzak discloses a traffic bearing structure with permeable pavement applying various aggregates, with California Bearing Ratio testing.

U.S. Pat. No. 8,337,117 to Vitale et al. discloses a composition for road construction with resilient modulus, FIG. 3, with percent California Bearing Ratio testing, FIG. 2, and less than 50 percent of coarse fraction No. 4 sieve passing through for the subbase.

Although some prior art teachings involve use of California Bearing Ratio (CBR), they does not show using an estimate of that value, whereas the present invention discloses methods of estimating CBR. The present invention differs from any previous work in that it uses multiple linear regression models to predict the California Bearing Ratio of a pavement subbase layer.

### BRIEF SUMMARY OF THE INVENTION

According to the present invention, estimation of the California Bearing Ratio of the subbase layer of flexible pavement is obtained by the application of multiple linear regression models. This approach is more efficient than repetitive CBR testing to measure soil strength of the subbase layer and results in a considerable cost saving.

In essence, the invention involves the method of applying a multiple linear regression model to a dataset of two-variables to six-variables for predicting the California Bearing Ratio (CBR) for a subbase layer intended to have an overlying surface of asphalt or Portland cement. Formulas involving multiple linear regression models with the following variables are applied:
(A) Percent of material retained on sieve size No. 4 (4.75 mm)
(B) Percent of material passing sieve size No. 4 and retained on sieve size No. 200 (0.075 mm)
(C) Percent of material passing sieve size No. 200
(L.A.) Los Angeles abrasion test for aggregate toughness and abrasion characteristics
(O.M.C.) Optimum Moisture Content
(Density) Soil density

During construction of new roads according to local specifications, samples were collected from different regions and tested for sieve analysis to determine: the percentage of aggregate retained on sieve No. 4 (A); percentage of aggregate passing sieve No. 4 and retained on sieve No. 200 (B); and percentage of material passing sieve No. 200 (C).

The A, B, and C factors were then tested to determine the relationship between moisture content and dry density. Finally each sample was prepared at optimum moisture content (O.M.C.) and at different density (maximum dry density among them), then tested to determine the CBR for each density.

Then, according to the invention, multiple linear regression models are applied to different datasets to relate the measured California Bearing Ratio (CBR) value to the different datasets, and preferably to the dataset comprising the percentage of optimum moisture content (OMC) contained in the subbase course, the Los Angeles abrasion test, and the density of the soil.

Regression analysis demonstrated that the density, optimum moisture content, and Los Angeles (L.A) value are the most effective parameters on CBR value among the others such as A, B and C (obtained from sieve analysis test). Regression analysis estimation indicated strong correlations (R²=0.94) between the L.A, OMC, and density values of the subbase layer. It was shown that the correlation equations obtained as a result of regression analyses are in satisfactory agreement with the test results, and the prediction model of the California Bearing Ratio (CBR) is fairly close to the corresponding actual results. The method of the invention is especially desirable for a preliminary design of a project where there are financial and time limitations.

In summation, the present invention generally comprises a method for predicting the California Bearing Ratio of a pavement subbase layer, comprising:

collecting samples from different regions of the subbase layer;

testing the samples to obtain a dataset of variables including at least moisture content and density;

preparing each sample at optimum moisture content and at different densities;

testing each sample to determine the California Bearing Ratio for each density; and

applying a multiple linear regression model to relate the determined California Bearing Ratio value to the dataset of variables to obtain a predicted value of the California Bearing Ratio of a subbase having comparable variables.

### BRIEF DESCRIPTION OF THE DRAWINGS

The foregoing, as well as other objects and advantages of the invention, will become apparent from the following detailed description when taken in conjunction with the accompanying drawings, wherein like reference characters designate like parts throughout the several views, and wherein:

FIG. 1 is a schematic isometric cut-away view of a typical asphalt concrete pavement.

FIG. 2 is a graph comparing the measured and predicted values of California Bearing Ratio.

FIG. 3 is a normal probability plot of the residuals.

FIG. 4 is a residual plot against predicted California Bearing Ratio.

FIG. 5 is a plot comparing the measured and predicted values of California Bearing Ratio.

### DETAILED DESCRIPTION OF THE PREFERRED EMBODIMENTS OF THE INVENTION

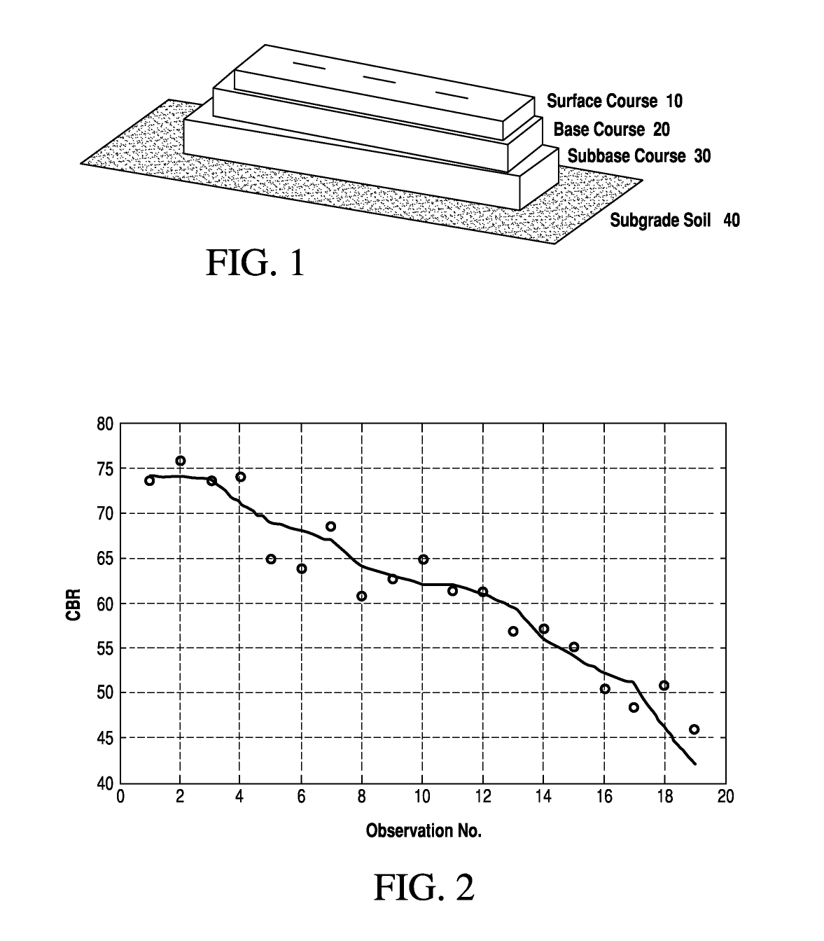

A typical pavement structure is shown schematically in FIG. 1, wherein a surface course 10 of asphalt, Portland cement, or the like, is placed on top of a base course 20 normally consisting of a mixture of aggregates such as sand, gravel, or crushed stone fastened together by cement. These may be compacted or stabilized by lime, Portland cement or asphalt. The base course is placed on top of a subbase course or layer 30 that usually comprises local aggregate materials that may consist of either unstabilized compacted aggregate or stabilized materials. The subbase course is placed on top of sub grade soil 40 that is the top surface of a roadbed upon which the pavement structure and shoulders are constructed. The purpose of the sub grade is to provide a platform for construction of the pavement and to support the pavement without undue deflection that would impact the pavement's performance. The upper layer of this natural soil may be compacted or stabilized to increase its strength.

New roads were constructed in the Makkah area of Saudi Arabia according to Ministry of Transportation specifications in Saudi Arabia for Gradation of the subbase layer (Tables 1 and 2). Samples were collected from different regions in the area during construction of the roads, and these samples were tested for sieve analysis to determine the percentage of aggregate retained on sieve number 4 (4.75 mm) (A), percentage of aggregate passing sieve number 4 and retained on sieve number 200 (0.075 mm) (B), and percentage of material passing sieve number 200 (C).

TABLE 1
M.O.T. specifications for Gradation of subbase layer.

| Sieve Size | M.O.T. specification limits |
|---|---|
| Designation | (% Passing) |
| 2″ | 100 |
| 1½″ | 90-100 |
| 1″ | 55-85 |
| ¾″ | 50-80 |
| ⅜″ | 40-70 |
| #4 | 30-60 |
| #10 | 20-50 |
| #40 | 10-30 |
| #200 | 0-15 |

TABLE 2
M.O.T. specifications for Quality requirements of subbase layer.

| | |
|---|---|
| Sand equivalent | 25 Min. |
| Plasticity index | 6 Max. |
| Abrasion loss | 50 Max. |
| CBR | 50 Min. |

The A, B and C factors were then tested to determine the relationship between moisture content and dry density. Finally, each sample was prepared at optimum moisture content (O.M.C.) and at different density (maximum dry density among them), then tested to determine the California Bearing Ratio (CBR) for each density.

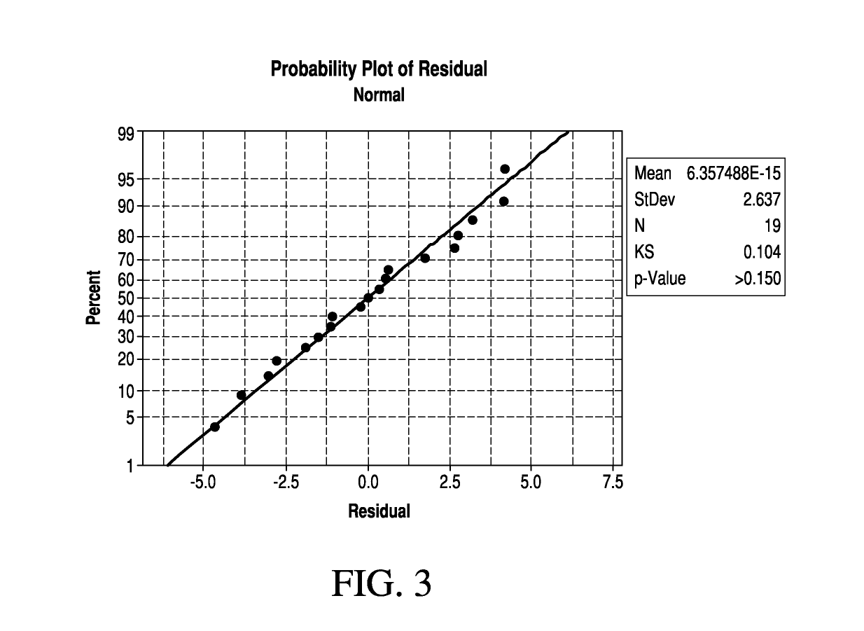

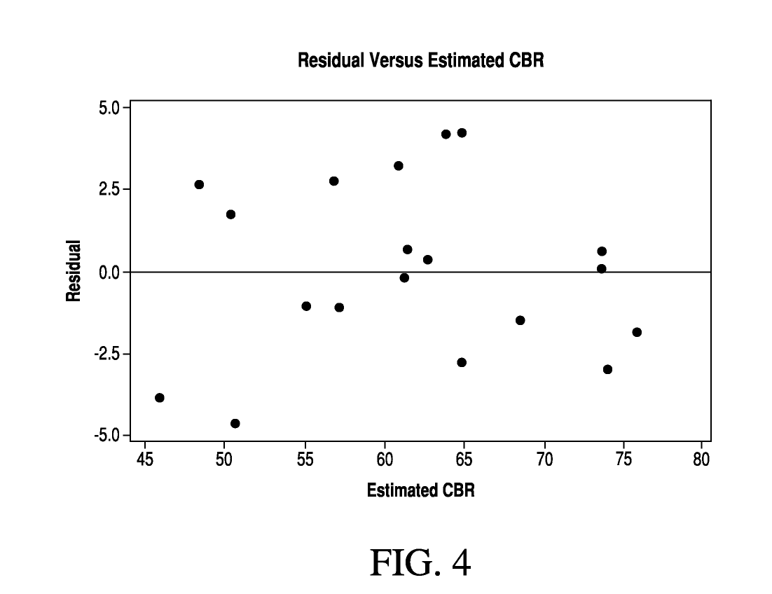

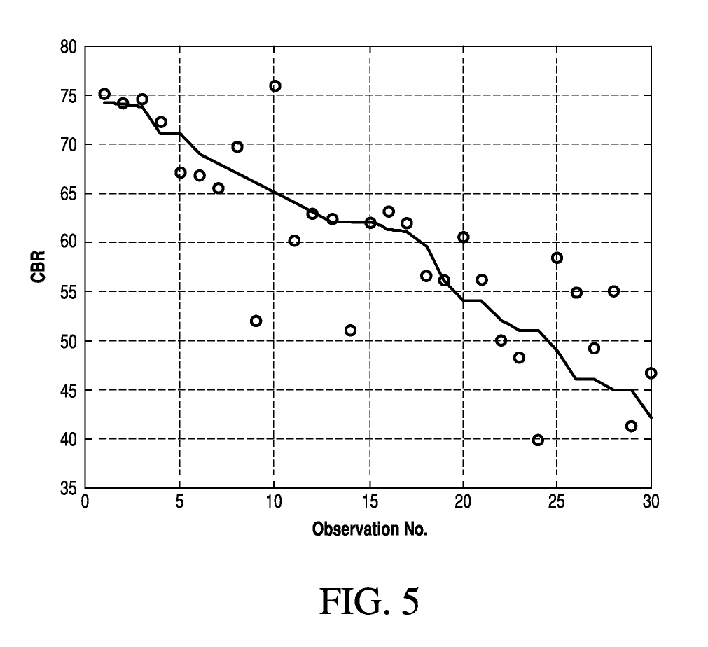

The regression model was used to relate the CBR value as measured by an expert soil engineer to service a subbase course of road structure to the OMC (percentage of optimum moisture content) contained in the subbase course, the Los Angeles abrasion test, and the density of soil. The relationships between the measured and predicted values of California Bearing Ratio are shown in FIG. 2. The normal probability plot of the residuals is shown in FIG. 3, and the residual plot against predicted California Bearing Ratio is shown in FIG. 4. FIG. 5 is a plot comparing the measured and predicted values of California Bearing Ratio.

The best fit model could be used for road design and estimation of the CBR of a subbase course layer in different roads—roads in the Makkah region based on the samples taken in this instance. Table 3 presents a sample of nineteen ideal measurements applied to nineteen subbase layers of the Makkah roads. The measurements include sieve analysis test parameters A %, B %, C %, the Los Angeles abrasion test, OMC, density, and CBR values. The CBR values are in the range between 74.2 to the minimum value of 49.

TABLE 3
Sample of 19 ideal subbase layers collected from different Makkah roads.

| | Sieve analysis test | | | Los- | | Density | |
|---|---|---|---|---|---|---|---|
| Observation | Parameters | | | Angeles | OMC | g/cm³ | CBR |
| No. | A % | B % | C % | % | % | % | % |
| 1 | 61.8 | 24.7 | 13.5 | 20.3 | 6.6 | 2.28 | 74.2 |
| 2 | 57 | 35 | 8 | 32 | 6.5 | 2.3 | 74 |
| 3 | 61.3 | 26.4 | 12.3 | 22.8 | 6.4 | 2.272 | 73.7 |
| 4 | 57 | 35 | 8 | 32 | 6.5 | 2.28 | 71 |
| 5 | 56.6 | 37.4 | 6 | 13.4 | 8.1 | 2.25 | 71 |
| 6 | 53.1 | 37.2 | 9.7 | 17 | 6.25 | 2.17 | 69 |
| 7 | 56.6 | 37.4 | 6 | 13.4 | 8.1 | 2.235 | 68 |
| 8 | 47.8 | 47.5 | 4.7 | 19 | 7.1 | 2.245 | 67 |
| 9 | 61.3 | 26.4 | 12.3 | 22.8 | 6.4 | 2.153 | 63 |
| 10 | 57 | 35 | 8 | 32 | 6.5 | 2.18 | 62 |
| 11 | 61.8 | 24.7 | 13.5 | 20.3 | 6.6 | 2.147 | 62 |
| 12 | 61.8 | 24.7 | 13.5 | 20.3 | 6.6 | 2.158 | 61.2 |
| 13 | 11.8 | 69.4 | 18.8 | 21 | 6.2 | 2.129 | 61 |
| 14 | 61.3 | 26.4 | 12.3 | 22.8 | 6.4 | 2.089 | 59.5 |
| 15 | 32 | 51.1 | 16.9 | 22 | 7.8 | 2.15 | 56 |
| 16 | 56.6 | 37.4 | 6 | 13.4 | 8.1 | 2.14 | 54 |
| 17 | 61.3 | 26.4 | 12.3 | 21.2 | 6.7 | 2.032 | 52.1 |
| 18 | 11.8 | 69.4 | 18.8 | 21 | 6.2 | 1.99 | 51 |
| 19 | 35 | 51 | 14 | 20.3 | 6.8 | 2.12 | 49 |

The general multiple linear regression model is given as below:

Yᵢ=β₀+β₁Xᵢ₁+β₂Xᵢ₂+ . . . +βₙXᵢₙ (1)

Where Xi=[Xᵢ₁,Xᵢ₂+ . . . +Xᵢₙ]^{T} is the vector of influencing factors in the data series, and β=[β₁,β₂, . . . βₙ] is the vector of the model's parameters. For instance, the regression model with six variables A %, B %, C %, Los Angeles, OMC and density is given as below:

CBRᵢ=β₀+β₁Aᵢ+β₂Bᵢ+β₃Cᵢ+β₄LosAngelesᵢ+β₅OMCᵢ+β₆Densityᵢ (2)

While this regression model is for six variables, all other possible regression models are considered.

The regression analysis calculates the Mean Square Error (MS_{E}) for each possible model. Since models with large MS_{E} are not likely to be selected as the best regression equations, it is necessary to examine details only of the models with small values of MS_{E}. Tables 4 to 6 list all possible regressions for the first nineteen observations listed in Table 3. The dataset are chosen such that the model also results in a minimum Mean Square Error for the P-Variable (MS_{E}(p)) and a high Coefficient of Multiple Determination (R p). In terms of R² improvement, there is little gain in going from a two-variable model to a six-variable model.

TABLE 4
All Possible Regression for the Data in Table 3 (No. of variables in the model is 1 and 2)

| No. of | | | | | | | | |
|---|---|---|---|---|---|---|---|---|
| variables in | | | | | | | | |
| Model | p | Variables in model | Rₚ² | SS_{R} (p) | SS_{E} (p) | MS_{E} (p) | R̅ₚ² | cₚ |
| 1 | 2 | A | 0.1368 | 219.3874 | 1384.8 | 81.4588 | 0.0860 | 2 |
| 1 | 2 | B | 0.1143 | 183.3218 | 1420.9 | 83.5803 | 0.0622 | 2 |
| 1 | 2 | C | 0.1030 | 165.1601 | 1439.0 | 84.6487 | 0.0502 | 2 |
| 1 | 2 | LosAngeles | 0.0965 | 154.8123 | 1449.4 | 85.2574 | 0.0434 | 2 |
| 1 | 2 | OMC | 0.0239 | 38.3102 | 1565.9 | 92.1104 | −0.0335 | 2 |
| 1 | 2 | Density | 0.8472 | 1359.1 | 245.0761 | 14.4162 | 0.8382 | 2 |
| 2 | 3 | A, B | 0.1454 | 233.2225 | 1371.0 | 85.6853 | 0.0386 | 3 |
| 2 | 3 | A, C | 0.1454 | 233.2225 | 1371.0 | 85.6853 | 0.0386 | 3 |
| 2 | 3 | A, LosAngeles | 0.2170 | 348.0976 | 1256.1 | 78.5056 | 0.1191 | 3 |
| 2 | 3 | A, OMC | 0.1511 | 242.4343 | 1361.8 | 85.1096 | 0.0450 | 3 |
| 2 | 3 | A, Density | 0.8488 | 1361.6 | 242.5935 | 15.1621 | 0.8299 | 3 |
| 2 | 3 | B, C | 0.1454 | 233.2225 | 1371.0 | 85.6853 | 0.0386 | 3 |
| 2 | 3 | B, LosAngeles | 0.1921 | 308.1415 | 1296.0 | 81.0029 | 0.0911 | 3 |
| 2 | 3 | B, OMC | 0.1255 | 201.2499 | 1402.9 | 87.6836 | 0.0161 | 3 |
| 2 | 3 | B, Density | 0.8513 | 1365.7 | 238.4686 | 14.9043 | 0.8328 | 3 |
| 2 | 3 | C, LosAngeles | 0.2038 | 326.9911 | 1277.2 | 79.8248 | 0.1043 | 3 |
| 2 | 3 | C, OMC | 0.1391 | 223.2166 | 1381.0 | 86.3107 | 0.0315 | 3 |
| 2 | 3 | C, Density | 0.8508 | 1364.9 | 239.2739 | 14.9546 | 0.8322 | 3 |
| 2 | 3 | LosAngeles, OMC | 0.0980 | 157.1586 | 1447.0 | 90.4393 | −0.0148 | 3 |
| 2 | 3 | LosAngeles, Density | 0.8472 | 1359.1 | 245.0558 | 15.3160 | 0.8281 | 3 |
| 2 | 3 | OMC, Density | 0.9220 | 1479.0 | 125.1538 | 7.8221 | 0.9122 | 3 |

CBR = California Bearing Ratio, %
O.M.C. = Optimum Moisture Content, %
A = % of aggregate retained on sieve No. 4 (4.75 mm), %
B = % of aggregate passing sieve No. 4 and retained on sieve No. 200 (0.075 mm), %
C = % of material passing sieve No. 200, %
L.A. = Los Angeles abrasion test, %
MS_{E} = mean square error
MS_{E} (p) = mean square error for the p-variable
R_{P}² = coefficient of multiple determination
SS_{R} (p) = regression sum of squares for the p-variable
SS_{E} (p) = error sum of squares for the p-variable
R̅ₚ² = adjusted Rₚ²
cₚ = total mean square error for the regression model

TABLE 5
All Possible Regression for the Data in Table 3 (No. of variables in the model is 3 and 4)

| No. of | | | | | | | | |
|---|---|---|---|---|---|---|---|---|
| variables in | | | | | | | | |
| Model | p | Variables in model | Rₚ² | SS_{R} (p) | SS_{E} (p) | MS_{E} (p) | R̅ₚ² | cₚ |
| 3 | 4 | A, B, C | 0.1454 | 233.2225 | 1371.0 | 91.3977 | −0.0255 | 4 |
| 3 | 4 | A, B, LosAngeles | 0.2319 | 372.0647 | 1232.1 | 82.1415 | 0.0783 | 4 |
| 3 | 4 | A, B, OMC | 0.1668 | 267.5215 | 1336.7 | 89.1111 | 0.0001 | 4 |
| 3 | 4 | A, B, Density | 0.8608 | 1380.8 | 223.3598 | 14.8907 | 0.8329 | 4 |
| 3 | 4 | A, C, LosAngeles | 0.2319 | 372.0647 | 1232.1 | 82.1415 | 0.0783 | 4 |
| 3 | 4 | A, C, OMC | 0.1668 | 267.5215 | 1336.7 | 89.1111 | 0.0001 | 4 |
| 3 | 4 | A, C, Density | 0.8608 | 1380.8 | 223.3598 | 14.8907 | 0.8329 | 4 |
| 3 | 4 | B, C, LosAngeles | 0.2319 | 372.0647 | 1232.1 | 82.1415 | 0.0783 | 4 |
| 3 | 4 | B, C, OMC | 0.1668 | 267.5215 | 1336.7 | 89.1111 | 0.0001 | 4 |
| 3 | 4 | B, C, Density | 0.8608 | 1380.8 | 223.3598 | 14.8907 | 0.8329 | 4 |
| 3 | 4 | A, LosAngeles, OMC | 0.2172 | 348.3863 | 125508 | 83.7201 | 0.0606 | 4 |
| 3 | 4 | A, LosAngeles, Density | 0.8488 | 1361.6 | 242.5439 | 16.1696 | 0.8186 | 4 |
| 3 | 4 | A, OMC, Density | 0.9220 | 1479.0 | 125.1444 | 8.3430 | 0.9064 | 4 |
| 3 | 4 | B, LosAngeles, OMC | 0.1921 | 308.1435 | 1296.0 | 86.4029 | 0.0305 | 4 |
| 3 | 4 | B, LosAngeles, Density | 0.8514 | 1365.7 | 238.4461 | 15.8964 | 0.8216 | 4 |
| 3 | 4 | B, OMC, Density | 0.9221 | 1479.2 | 124.9723 | 8.3315 | 0.9065 | 4 |
| 3 | 4 | C, LosAngeles, OMC | 0.2093 | 335.7777 | 1268.4 | 84.5606 | 0.0512 | 4 |
| 3 | 4 | C, losAngeles, Density | 0.8509 | 1365.0 | 239.1813 | 15.9454 | 0.8211 | 4 |
| 3 | 4 | C, OMC, Density | 0.9239 | 1482.0 | 122.1467 | 8.1431 | 0.9086 | 4 |
| 3 | 4 | LosAngeles, OMC, Density | 0.9405 | 1508.7 | 95.4914 | 6.3661 | 0.9286 | 4 |
| 4 | 5 | A, B, C, LosAngeles | 0.2319 | 372.0647 | 1232.1 | 88.0088 | 0.0125 | 5 |
| 4 | 5 | A, B, C, OMC | 0.1668 | 267.5215 | 1336.7 | 95.4761 | −0.0713 | 5 |
| 4 | 5 | A, B, C, Density | 0.8608 | 1380.8 | 223.3598 | 15.9543 | 0.8210 | 5 |
| 4 | 5 | A, B, LosAngeles, OMC | 0.2336 | 374.7542 | 1229.4 | 87.8167 | 0.0146 | 5 |
| 4 | 5 | A, B, LosAngeles, Density | 0.8610 | 1381.2 | 222.9416 | 15.9244 | 0.8213 | 5 |
| 4 | 5 | A, C, LosAngeles, OMC | 0.2336 | 374.7542 | 1229.4 | 87.8167 | 0.0146 | 5 |
| 4 | 5 | B, C, LosAngeles, OMC | 0.2336 | 374.7542 | 1229.4 | 87.8167 | 0.0146 | 5 |
| 4 | 5 | B, C, LosAngeles, Density | 0.8610 | 1381.2 | 222.9416 | 15.9244 | 0.8213 | 5 |
| 4 | 5 | A, C, LosAngeles, Density | 0.8610 | 1381.2 | 222.9416 | 15.9244 | 0.8213 | 5 |
| 4 | 5 | A, LosAngeles, OMC, Density | 0.9409 | 1509.4 | 94.7709 | 6.7693 | 0.9240 | 5 |

TABLE 6
All Possible Regression for the Dataset in Table 3 (No. of variables in the model is 5 and 6).

| No. of | | | | | | | | |
|---|---|---|---|---|---|---|---|---|
| variables in | | | | | | | | |
| Model | p | Variables in model | Rₚ² | SS_{R} (p) | SS_{E} (p) | MS_{E} (p) | R̅ₚ² | cₚ |
| 5 | 6 | A, B, C, LosAngeles, OMC | 0.2336 | 374.7542 | 1229.4 | 94.5718 | −0.0612 | 6 |
| 5 | 6 | A, B, C, LosAngeles, Density | 0.8610 | 1381.2 | 222.9416 | 17.1494 | 0.8076 | 6 |
| 5 | 6 | A, B, C, OMC, Density | 0.9250 | 1483.9 | 120.3168 | 9.2551 | 0.8962 | 6 |
| 5 | 6 | A, B, LosAngeles, OMC, Density | 0.9460 | 1517.5 | 86.6819 | 6.6678 | 0.9252 | 6 |
| 5 | 6 | A, C, LosAngeles, OMC, Density | 0.9460 | 1517.5 | 86.6819 | 6.6678 | 0.9252 | 6 |
| 5 | 6 | B, C, LosAngeles, OMC, Density | 0.9460 | 1517.5 | 86.6819 | 6.6678 | 0.9252 | 6 |
| 6 | 7 | A, B, C, LosAngeles, OMC, Density | 0.9460 | 1517.5 | 86.6819 | 7.2235 | 0.9189 | 7 |

The best two-variable model is (OMC, Density), and the best three-variable model is (Los Angeles, OMC, Density). The minimum values of MS_{E}(p) occur for the three-variable model (Los Angeles, OMC, Density). While there are several other models that have relatively small values of MS_{E}(p), such as (A, B, Los Angeles, OMC, Density), and (A, Los Angeles, OMC, Density), the model (Los Angeles, OMC, Density) is superior with respect to the MS_{E}(p) criterion. This model also maximizes the adjusted coefficient of multiple determination ( R̅ₚ²). Since this model results in a minimum MS_{E}(p) and a high R²ₚ, it is selected as the “best regression equation. The final model is:

CBR=−112.4335−0.2856LosAngeles−4.7280OMC+98.4613Density (3)

FIG. 2 compares the measured CBR values with their corresponding predicted values. The normal probability plot of the residuals is shown in FIG. 3. Since the residuals fall approximately along a straight line in FIG. 3, it is concluded that there is no severe departure from normality. The residuals are also plotted against predicted CBR in FIG. 4.

Testing and verification of the model (Los Angeles, OMC, Density) was conducted using a test dataset of thirty observations. The CBR is in the range between 74.2% and 42%. The predicted CBR values were calculated using equation (3). The plot in FIG. 5 compares the CBR values with predicted values. The resulting MSE is 9.8742%.

The prediction model of California Bearing Ratio is fairly close to the corresponding actual results. Regression analysis was performed and it was found that the density, optimum moisture content, and Los Angeles (L.A.) values are the most effective parameters on CBR value among the others such as A, B and C (obtained from sieve analysis tests). Regression analysis estimation indicated strong correlations (R²=0.94) between the L.A., OMC and Density values of the subbase layer. It was shown that the correlation equations obtained as a result of regression analyses are in satisfactory agreement with the test results. The proposed correlations will therefore be practical for a preliminary design of a project where there are financial and time limitations.

What is claimed is:

1. A method for predicting the California Bearing Ratio of a pavement subbase layer, comprising:
collecting samples from different regions of the subbase layer;
testing the samples to obtain a dataset of variables including at least moisture content and density;
preparing each sample at optimum moisture content and at different densities;
testing each sample to determine the California Bearing Ratio for each density; and
applying a multiple linear regression model to relate the determined California Bearing Ratio value to selected variables from the dataset to obtain a predicted value of the California Bearing Ratio of a subbase having comparable variables.

2. The method claimed in claim 1, wherein:
the dataset of variables is selected from the group consisting of: percent of material retained on sieve size No. 4; percent of material passing sieve size No. 4 and retained on sieve size No. 200; percent of material passing sieve size No. 200; Los Angeles abrasion test for aggregate toughness and abrasion characteristics; percentage of optimum moisture content in the subbase; and soil density.

3. The method of claim 2, wherein:
the selected variables from the dataset comprises the percentage of optimum moisture content in the subbase, the Los Angeles abrasion test, and soil density.

4. The method of claim 2, wherein:
the selected variables from the dataset comprises the percentage of optimum moisture in the subbase, the Los Angeles abrasion test, soil density, and percent of material retained on sieve size No. 4.

5. The method of claim 2, wherein:
the selected variables from the dataset comprises the percentage of optimum moisture in the subbase, the Los Angeles abrasion test, soil density, percent of material retained on sieve size No. 4, and percent of material passing sieve size No. 4 and retained on sieve size No. 200.

6. The method of claim 2, wherein:
the selected variables from the dataset comprises the percentage of optimum moisture in the subbase, the Los Angeles abrasion test, soil density, percent of material retained on sieve size No. 4, percent of material passing sieve size No. 4 and retained on sieve size No. 200, and percent of material passing sieve No. 200.

7. The method of claim 2, wherein:
the selected variables from the dataset comprises soil density.

8. The method of claim 2, wherein:
the selected variables from the dataset comprise soil density and percent of material retained on sieve No. 4.

9. The method of claim 2, wherein:
the selected variables from the dataset comprise soil density and percent of material passing sieve size No. 4 and retained on sieve size No. 200.

10. The method of claim 2, wherein:
the selected variables from the dataset comprise soil density and percent of material passing sieve size No. 200.

11. The method of claim 2, wherein:
the selected variables from the dataset comprise the Los Angeles abrasion test, and soil density.

12. The method of claim 2, wherein:
the selected variables from the dataset comprise percentage of optimum moisture content in the subbase and soil density.

13. The method of claim 2, wherein: the general multiple linear regression model is given as Yᵢ=β₀+β₁Xᵢ₁+β₂Xᵢ₂+ . . . +βₙXᵢₙ.

14. The method of claim 2, wherein: the multiple linear regression model for six variables is given as CBRᵢ=β₀+β₁Aᵢ+β₂Bᵢ+β₃Cᵢ+β₄LosAngelesᵢ+β₅OMCᵢ+β₆Densityᵢ.
